(19) United States
(12) Patent Application Publication
(10) Pub. No.: US 2014/0276606 A1
(43) Pub. Date: Sep. 18, 2014

(54) MOVEABLE CUFF

(71) Applicant: Cook Medical Technologies LLC, Bloomington, IN (US)

(72) Inventor: Meridith Cavett, Dexter, MI (US)

(21) Appl. No.: 14/203,671

(22) Filed: Mar. 11, 2014

Related U.S. Application Data
(60) Provisional application No. 61/786,872, filed on Mar. 15, 2013.

(51) Int. Cl. A61M 25/04

(52) U.S. Cl. CPC A61M 25/04
USPC 604/508; 604/175

(57) ABSTRACT

Disclosed is a catheter assembly that includes a catheter, a sheath on the catheter, a sleeve on the sheath and a tissue ingrowth cuff affixed to the sleeve. The sleeve and sheath are movable along the length of the catheter with the sheath being removable from between the sleeve and the catheter. When the sheath is removed, the sleeve becomes substantially fixed in position on the catheter. The sheath may have a comparatively low coefficient of friction against the catheter while the sleeve may have a comparatively high coefficient of friction against the catheter.

### CROSS REFERENCE TO RELATED APPLICATIONS

The present application claims the benefit of U.S. Provisional Patent Application Ser. No. 61/786,872 filed Mar. 15, 2013, which is hereby incorporated by reference.

### BACKGROUND

This disclosure relates to catheter cuffs, and more specifically to catheter cuffs whose position can be relocated by an operator.

Many types of catheters are indicated for short or long term access into a body. In the context of human medicine this includes monitoring of internal pressure, e.g., pulmonary blood pressure, blood sampling, nutrition and administration of drugs and fluids.

One type of catheter that is used for long term access is a tunneled catheter that passes through a length of subcutaneous tissue prior to entering the site to which access is desired, e.g., a blood vessel. This can be done by first gaining access to the desired site, e.g., the internal jugular or subclavian veins, via an incision through the skin. A second incision site through the skin is then created spaced apart from the first access site. The catheter is then tunneled through the subcutaneous tissue between the second and first incision sites and is inserted into the desired location in the body. This locates the external portion of the catheter away from the first access site.

In some cases, the catheter includes a tissue ingrowth body on the external surface of the catheter to promote subcutaneous tissue to incorporate around the ingrowth body to fix the catheter in place and to block pathogens from migrating along the length of the catheter into the body. For example, a fiber cuff can be glued to the catheter shaft 1 cm to 13 cm away from the catheter manifold.

Permanently gluing the cuff in place on the catheter during manufacture limits the applications an individual catheter can be employed for and places limitations on the use of the catheter as the length of tunneling will be dictated by the amount of catheter remaining after placement in the desired site in the body, and a physician cannot always accurately predict how much catheter length will be required to reach a desired location. Once properly located, the physician will not want to move the tip of the catheter, and a physician will not want to change out an inserted catheter with a new catheter with a different cuff location to accommodate tunneling.

### SUMMARY

Disclosed is a catheter assembly that includes a catheter, a sheath on the catheter, a sleeve on the sheath and a tissue ingrowth cuff affixed to the sleeve. The sleeve and sheath are movable along the length of the catheter with the sheath being removable from between the sleeve and the catheter. When the sheath is removed, the sleeve becomes substantially fixed in position on the catheter. The sheath may have a comparatively low coefficient of friction against the catheter while the sleeve may have a comparatively high coefficient of friction against the catheter.

The disclosed catheter assembly may include a catheter with an external surface that is substantially smooth and free of projections. The sleeve may be resilient and have a neutral internal diameter dimension that is smaller than the outer diameter of the catheter, requiring the sleeve to be expanded to fit over the catheter. The sleeve may be constructed of silicone. The sleeve may form a substantially fluid impenetrable seal around the external surface of the catheter after the sheath is removed. The sheath may be a peel away sheath, allowing the sheath to be removed from the catheter assembly when it is removed from between the sleeve and the catheter.

The disclosed catheter assembly described above may be included as part of a kit for a surgeon or other medical professional to use to insert the disclosed catheter.

### BRIEF DESCRIPTION OF THE DRAWINGS

FIG. 1 illustrates a perspective view of a catheter assembly including a catheter, a sheath, and a tissue ingrowth cuff attached to a sleeve.

FIG. 2 illustrates a cross-section of FIG. 1 along line 2-2.

FIG. 3 illustrates a perspective view of the FIG. 1 catheter assembly showing longitudinal movement of the sheath and tissue ingrowth cuff along the long axis of the catheter.

FIG. 4 illustrates a perspective view of the FIG. 1 catheter assembly with the sheath and tissue ingrowth cuff positioned on the catheter.

FIG. 5 illustrates a perspective view of the FIG. 4 configuration with the sheath partially removed.

FIG. 6 illustrates a perspective view of the FIG. 4 configuration with the sheath removed.

FIG. 7 illustrates a cross-section of FIG. 6 along line 7-7.

FIG. 8 illustrates an alternative embodiment of the FIG. 1 catheter assembly incorporating an alternative sheath configuration.

### DETAILED DESCRIPTION

Reference will now be made to certain embodiments and specific language will be used to describe the same. It should be understood that no limitation of the scope of this disclosure and the claims are thereby intended, such alterations, further modifications and further applications of the principles described herein being contemplated as would normally occur to one skilled in the art to which this disclosure relates. In several figures, where there are the same or similar elements, those elements are designated with the same or similar reference numerals.

The disclosed system features a catheter cuff positioning system comprising a catheter tube, a movable cuff assembly and sheath. This positioning system allows the physician to place a cuff along the long axis of the catheter after insertion of the catheter into the patient. The operator does not need to pre-determine where the cuff is to be located prior to surgery.

As contemplated herein, embodiments of the disclosed system can include a cuff positioning system useful in conjunction with a thin-walled catheter, a dialysis catheter, a high-flow catheter, a high pressure catheter, or a tunneled central catheter as non-limiting examples. The skilled artisan will recognize that the disclosed system can be used with other type of catheters in addition to those listed above.

Catheters such as thin-walled catheters, high-flow catheters, and high pressure catheters generally include at least one lumen usable to pass fluids into or out of a patient's body. The disclosed system should not be understood to be limited to the number of lumens present in the catheter. Catheters with different numbers of lumens are used for different purposes, as is known to the skilled artisan. Moreover, embodiments of the disclosed system may include other lumen configurations and cross sections, such as multiple lumens of different sizes and shapes. Furthermore, the disclosed system may also be used with catheters or other devices that have no lumen.

Referring to the drawings, FIGS. 1 and 2 illustrate an embodiment of catheter assembly 20. Catheter assembly 20 includes catheter 22, sheath 40, sleeve 60 and cuff 80. Catheter 22 has external surface 24 and defines lumen 26, lumen 28, port 30 and port 32. Catheter 22 also includes connectors 34 and 36. Connector 34 is fluidly connected to port 30 via lumen 26. Connector 36 is fluidly connected to port 32 via lumen 28. Connectors 34 and 36 are luer locks in the illustrated system configured to connect external tubing to catheter 22. However, connectors 34 and 36 may be any type of appropriate connector, including non-fluid connections where appropriate. For example, an optical connection may be used in conjunction with an optical sensor.

Sheath 40 is a peel-away sheath. Sheath 40 defines lumen 42 and includes internal surface 44, external surface 46, score line 48 and pull tabs 50 and 52. Sheath 40 is positioned between sleeve 60 and catheter 22. Removal of sheath 40 leaves sleeve 60 firmly engaged on catheter 22.

Sleeve 60 defines lumen 62 and includes internal surface 64 and external surface 66. Sleeve 60 may be a cylindrical member of silicone or other bio-compatible polymer. Sleeve 60 is constructed of a material that can elastically deform in the radial hoop direction. Sleeve 60 has a neutral (i.e. unstressed) internal diameter dimension smaller than the outer diameter of catheter 22 and the outer diameter of sheath 40. Sleeve 60 exerts a radial compression force when elastically expanded in the radial hoop direction to cover catheter 22 and sheath 40, as noted below.

Cuff 80 is a cylindrically shaped cuff of tissue-ingrowth material that promotes ingrowth of tissue when implanted in bodily tissue, such as under a patient's skin. Cuff 80 is attached to sleeve with adhesive 82 in this embodiment. Adhesive 82 may be a bio-compatible adhesive. Alternatively, cuff 80 may be attached to sleeve 60 by any other known mechanism, including, but not limited to mechanical attachments such as suture or staples. Sleeve 60 with cuff 80 attached define cuff assembly 90.

Catheter assembly 20 is assembled by inserting catheter 22 in lumen 42 of sheath 40. Sleeve 60 is expanded in a radial direction and sheath 40 is inserted into lumen 62 then sleeve 60. Cuff 80 is then glued onto sleeve 60 with adhesive 82. In one embodiment, sleeve 60 is constructed of silicone tubing which can be soaked to swell the tubing to allow sleeve 60 to be placed in position on sheath 40. Suitable solvents to swell the silicone cylindrical member include chlorinated hydrocarbons such as methylene chloride and perchloroethylene, perfluorohydrocarbons, hydrochlorofluorocarbons, and Novec®.

Internal surface 44 of sheath 40 and external surface 24 are constructed and arranged such that sheath 40 is slidable on catheter 22. In this regard, internal surface 44 may have a comparatively low coefficient of friction against external surface 24. Cuff assembly 90 positioned on sheath 40 and catheter 22 defines movable cuff assembly 100. Conversely, internal surface 64 of sleeve 60 is constructed and arranged to be substantially fixed in position on external surface 24 of catheter 22. In this regard, internal surface 64 may have a comparatively high coefficient of friction against external surface 24.

Movable cuff assembly 100 may be moved along the length of catheter 22 over external surface 24. As shown in FIG. 3, movable cuff assembly is repositionable, e.g. from position X to position X′ or vice versa.

Sheath 40 may be removed from between sleeve 60 and catheter 22 to substantially fix cuff assembly 90 in position on catheter 22. Sheath 40 may be a peel-away introducer sheath. By pulling on pull tabs 50 and 52, sheath 40 may separate along score line 48. Pulling on pull tabs 50 and 52 advances cuff assembly 90 off sheath 40 onto catheter 22. In this way, cuff assembly 90 can be located in a particular position on catheter 22 after catheter 22 has been inserted into a patient and a particular position for cuff assembly 90 can be determined based on a particular patient's anatomy and the actual position of catheter 22 when inserted into the patient.

FIGS. 4, 5 and 6 illustrate the removal of sheath 40 positioning cuff assembly 90 on catheter 22. FIG. 4 illustrates movable cuff assembly 100 positioned near the desired location for cuff assembly 90 on catheter 22. Once positioned, an operator separates sheath 40 along score line 48 by pulling on pull tabs 50 and pushes cuff assembly 90 off sheath 40. This is shown in FIG. 5. Once sheath 40 is completely separated along score line 48 and removed from between cuff assembly 90 and catheter 22, cuff assembly 90 is substantially fixed in position on catheter 22 as shown in FIGS. 6 and 7.

FIG. 8 illustrates catheter assembly 120 that is similar to catheter assembly 20 but for using sleeve 140 instead of sleeve 40. Sleeve 140 is an alternate embodiment of sleeve 40 that includes a fold-over near pull tabs 150 and 152 that changes the angle that pull tabs 150 and 152 extend from sleeve 140. Otherwise, sleeve 140 and catheter assembly 120 function in the same way described above with regard to catheter assembly 20.

It will be understood that catheter assembly 20, 120 or parts thereof as described previously may be provided as part of a kit for a surgeon or other medical professional. Such a kit may include, for example, a catheter, a sheath, a sleeve, and a tissue ingrowth cuff in any of the embodiments noted above. A kit may also include additional tools (e.g. introducer(s) or needle(s)) useful for inserting or using assembly 20, 120.

This disclosure serves to illustrate and describe what is claimed below to aid in the interpretation of the claims. However, this disclosure is not restrictive in character because not every embodiment covered by the claims is necessarily illustrated and described. All changes and modifications that come within the scope of the claims are desired to be protected, not just those embodiments explicitly described.

We claim:

1. A catheter assembly comprising:
a catheter including an external surface and having an outer diameter;
a sheath defining a first lumen, wherein the catheter passes through the first lumen and wherein the sheath has a comparatively low coefficient of friction against the external surface of the catheter;
a sleeve defining a second lumen, wherein the sheath passes through the second lumen and wherein the sleeve has a comparatively high coefficient of friction against the external surface of the catheter; and
a tissue ingrowth cuff affixed around the circumference of the sleeve, wherein the sleeve and sheath are movable over the external surface of the catheter, wherein the sheath is constructed and arranged to be selectively removable from between the sleeve and the catheter and wherein the sleeve is constructed and arranged to be substantially fixed in position on the external surface of the catheter after the sheath is removed from between the sleeve and the catheter.

2. The catheter assembly of claim 1, wherein the external surface of the catheter is substantially smooth and free of projections.

3. The catheter assembly of claim 1, further comprising an adhesive affixing the tissue ingrowth cuff to the sleeve.

4. The catheter assembly of claim 1, wherein the sleeve is resilient and has a neutral internal diameter dimension smaller than the outer diameter of the catheter.

5. The catheter assembly of claim 1, wherein the sleeve is expanded to increase its internal diameter when the sleeve is positioned around the sheath.

6. The catheter assembly of claim 1, wherein the sleeve is comprised of silicone.

7. The catheter assembly of claim 1, wherein the sheath comprises a non-longitudinally weakened area along the axial length of the sheath.

8. The catheter assembly of claim 1, wherein the sheath is a peel away sheath.

9. The catheter assembly of claim 1, wherein the sleeve is constructed and arranged to form a substantially fluid impenetrable seal around the external surface of the catheter after the sheath is removed from between the sleeve and the catheter.

10. A kit comprising:
a catheter including an external surface and having an outer diameter;
a sheath defining a first lumen larger than the outer diameter of the catheter, wherein the sheath has a comparatively low coefficient of friction against the external surface of the catheter;
a sleeve defining a second lumen, wherein the sheath passes through the second lumen with the sleeve positioned around the sheath and wherein the sleeve has a comparatively high coefficient of friction against the external surface of the catheter; and
a tissue ingrowth cuff affixed around the circumference of the sleeve, wherein the sheath is constructed and arranged to receive the catheter in a slidable relationship, wherein the sheath is constructed and arranged to be selectively removable from the sleeve and wherein the sleeve is constructed and arranged to be substantially fixed in longitudinal position on the external surface of the catheter after the sheath is removed from the sleeve.

11. The kit of claim 10, wherein the external surface of the catheter is substantially smooth and free of projections.

12. The kit of claim 10, further comprising an adhesive affixing the tissue ingrowth cuff to the sleeve.

13. The kit of claim 10, wherein the sleeve is resilient and has a neutral internal diameter dimension smaller than the outer diameter of the catheter and wherein the sleeve is biased to increase its internal diameter when the sleeve is positioned around the sheath.

14. The kit of claim 10, wherein the sleeve is comprised of silicone.

15. The kit of claim 10, wherein the sheath comprises a non-longitudinally weakened area along the axial length of the sheath.

16. The kit of claim 10, wherein the sheath is a peel away sheath.

17. The kit of claim 10, wherein the sleeve is constructed and arranged to form a substantially fluid impenetrable seal around the external surface of the catheter when the sleeve is in contact with the external surface of the catheter.

18. A method of uniting an apparatus, the method comprising:
for a particular clinical application for a particular patient, determining a longitudinal location on a catheter to position a tissue ingrowth cuff;
moving a sheath longitudinally along the catheter, wherein the sheath is positioned around the catheter, wherein the sheath carries a sleeve positioned around the sheath and wherein the sleeve includes the tissue ingrowth cuff affixed around the circumference of the sleeve, wherein the sleeve has a comparatively high coefficient of friction against the catheter;
removing the sheath from between the sleeve and the catheter thereby longitudinally positioning the sleeve and the tissue ingrowth cuff on the catheter.

19. The method of claim 18, further comprising:
before determining a longitudinal location on the catheter to locate the tissue ingrowth cuff, inserting the catheter into the particular patient.

20. The method of claim 18, further comprising:
after moving the sheath longitudinally along the catheter, separating the sheath into two parts that are removable from between the sleeve and the catheter.

21. The method of claim 18, wherein the sleeve is resilient and has a neutral diameter dimension smaller than an outer diameter of the catheter.
